(19) United States
(12) Patent Application Publication
(10) Pub. No.: US 2017/0258544 A1
(43) Pub. Date: Sep. 14, 2017

(54) MINIMALLY INVASIVE SPINE SURGERY DRAPE

(71) Applicant: Said G. Osman, Frederick, MD (US)

(72) Inventor: Said G. Osman, Frederick, MD (US)

(21) Appl. No.: 15/068,052

(22) Filed: Mar. 11, 2016

(51) Int. Cl. A61B 46/23; A61B 46/00; A61F 7/00

(52) U.S. Cl. CPC A61B 46/23; A61F 7/00; A61B 46/40; A61B 2046/201

(57) ABSTRACT

A surgical drape includes a center section having an operating window and extending longitudinally from a head end to a foot end, and a side section positioned on each longitudinal side of the center section. The side sections may be at least partially transparent. An elastic band extends through outermost ends of the side sections and across the foot end of the center section, and at least one surgical fluid collection pocket is disposed adjacent the operating window. Additional features of the drape may include a warming bladder and/or an extension section that is expandable to cover a machine adjacent the drape, such as an x-ray fluoroscope machine.

### CROSS-REFERENCES TO RELATED APPLICATIONS

(NOT APPLICABLE)

### STATEMENT REGARDING FEDERALLY SPONSORED RESEARCH OR DEVELOPMENT

(NOT APPLICABLE)

### BACKGROUND OF THE INVENTION

The invention relates to surgical drapes and, more particularly, to a surgical drape including characteristics and functional components that promote a sterile surgical field, improve the surgeon's field of view, and increase patient comfort.

Conventional surgical drapes are made of opaque fabrics, which may be made of cloth or synthetic materials. After sterile preparation of the operative site on the patient, the sterile drape is placed on the patient to cover the non-sterile part of the body, leaving only the surgical site exposed. Most of the surgical tools needed by the surgeon are placed either on a surgical tray, or in a sterile pocket, which may be part of the drape or come as its accessory. For open surgical procedures, there are only few tools that need to be on the floor, for example foot pedals for the surgical bur, drills, or electro-cautery. For the minimally invasive surgical procedures, in areas such as the spine, in addition to the noted foot operated tools, the surgeon needs ancillary tools such as an image intensifier. When visualizing a body structure with the images being taken parallel to the floor, a portion of the intensifier, the so called C-arm, has to be placed in a horizontal position—under the operating table (typically) or in an over-the-top position (less frequently). To keep the surgical field sterile, the C-Arm, which projects on either side of the table, must be kept sterile and thus typically requires an additional drape over one of the protruding ends (usually on the side of the surgeon) of the image intensifier. In this horizontal position, an opaque drape over the image intensifier makes it impossible for the surgeon to see the image intensifier or the tools on the floor.

Pulling the foot-operated tools away from underneath the drapes for the surgeon to visualize them often creates yet another problem, where the foot pedal may be too far from the patient to permit performance of the procedure, with the image intensifier in the horizontal position. Often this leads to an awkward situation whereby an assistant has to place the surgeon's foot on the tool for him/her to perform surgery with the image intensifier in this horizontal position.

Additionally, there may be important devices that are suspended under the operating table, including but not limited to, Foley catheter tubes and neuromonitor wires. The patient's belly may be protruding below the frame on which the patient is placed. Turning the C-Arm from the vertical to the horizontal position may pull against these devices, or cause injury to the patient. Hence, an assistant and/or the radiography technician has to look underneath the drapes to make sure these structures are not impacted by the C-Arm.

Furthermore, the endoscopic procedures utilize several hand-operated tools, including the endoscope, irrigation fluid system, light cable, video-camera, suction tubes, cautery cables, and cables of various automated tools. The multiplicity of cables and tubes can lead to entanglement and crowding of these structures on the surgical field, making the procedure more difficult.

### BRIEF SUMMARY OF THE INVENTION

According to preferred embodiments, a surgical drape may be made for different parts of the patient's anatomy. In one embodiment, the surgical drape includes an opaque portion that covers the body and transparent portions attached to sides of the opaque central portion. There are accessory transparent components which may be attached to the main drape as needed, to maintain a sterile surgical field. Additionally, the drape may include pockets for collection of irrigation fluid and pockets for arrangement of the various operative tools on the surgical field. When the patient is draped, the surgeon and the operating room staff will be able to see through the drape as the image intensifier is moved from the vertical to horizontal position, thereby enabling them to monitor the safety of the aforementioned tools and the patient. The surgeon will be able to see the foot-operated instruments on the floor through the drape thus obviating the need for the assistants to guide his/her foot to the instruments. Arrangements of the various cables and tubes in designated pockets allow orderly placement on the operative field thus preventing or minimizing entanglement of the devices on the surgical field.

In an exemplary embodiment, a surgical drape includes a center section having an operating window and extending longitudinally from a head end to a foot end, and a side section positioned on each longitudinal side of the center section. The side sections may be at least partially transparent. An elastic band extends through outermost ends of the side sections and across the foot end of the center section, and at least one surgical fluid collection pocket is disposed adjacent the operating window. A warming bladder may be positioned adjacent the center section.

The side sections may be connected to the center section by any currently practiced method including, without limitation, sewing, gluing or a hook and loop fastener. The center section may be T-shaped including an upper body component and a lower body component. The warming bladder may include an upper body bladder positioned adjacent the upper body component of the center section and a lower body bladder positioned adjacent the lower body component of the center section. The upper body component may include an arm extension drape. The upper part of side section may be expandable and due to the elasticated margin acts as a pocket, covering the fluoroscopy machine in horizontal position, thus preserving a sterile surgical field.

In another embodiment, an expandable pocket may be securable to, or be part of, at least one of the side sections, where the expandable pocket is sized to cover a machine such as a fluoroscopy machine adjacent the surgical drape. The drape may also include an equipment manifold attachable to the center section or the side sections. The equipment manifold may include a first plurality of compartments for receiving surgical tubes and cables and a second plurality of compartments for receiving surgical tools. The manifold may include a first level with the first plurality of compartments and a second level on top of the first level with the second plurality of compartments. The drape may also include one part of a removable fastener secured to at least of the center section and the side sections, where the equipment manifold may include another part of the removable fastener secured on an underside of the equipment manifold and selectively attachable to the one part of the removable fastener.

In another exemplary embodiment, a surgical drape includes a center section having an operating window and extending longitudinally from a head end to a foot end, and a side section positioned on each longitudinal side of the center section. The side sections may be at least partially transparent. At least one surgical fluid collection pocket is disposed adjacent the operating window. A partially or completely transparent side extension drape may be connected to the center section that may be expandable to cover a (fluoroscopy) machine adjacent the surgical drape.

### BRIEF DESCRIPTION OF THE DRAWINGS

These and other aspects and advantages will be described in detail with reference to the accompanying drawings, in which:

FIG. 1 is a plan view of the surgical drape;

FIG. 2 is a close-up view of an operating window in the surgical drape;

FIG. 3 shows an alternative embodiment of the surgical drape; and

FIGS. 4-6 show variations on pocket openings in the drape for accommodating an X-ray fluoroscope.

### DETAILED DESCRIPTION OF THE INVENTION

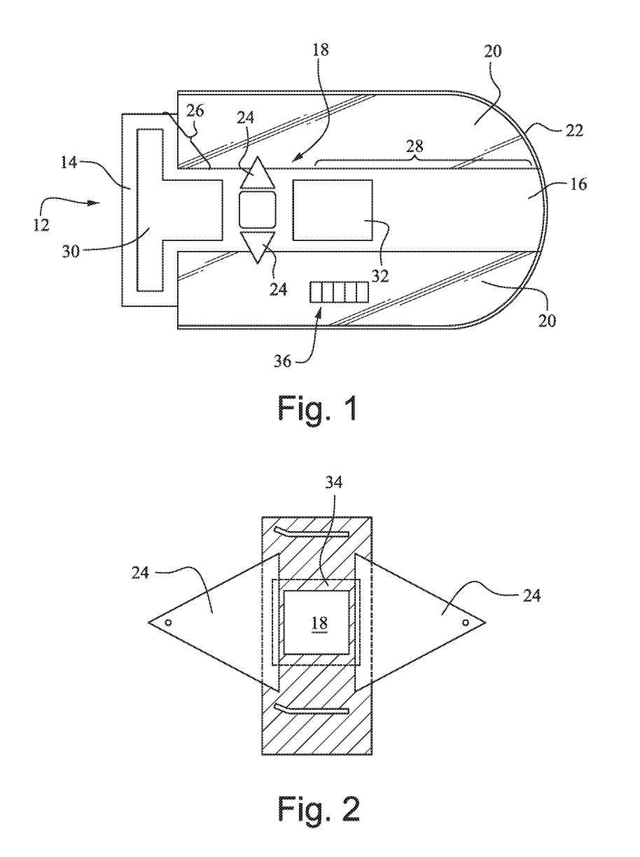

FIG. 1 is a plan view of the surgical drape according to a preferred embodiment. The drape includes a center section 12 extending longitudinally from a head end 14 to a foot end 16. This center section includes an operating window 18 and at least one surgical fluid collection pocket 24 disposed adjacent the operating window 18. Preferably, the center section 12 is formed of an opaque material.

A side section 20 is positioned on each longitudinal side of the center section 12 as shown. Preferably, the side sections are at least partially transparent. The center section 12 may be made out of a single sheet of material such as cloth or other appropriate fabric, or cut separately and attached together by known methods. The side sections 20 may be secured to the center section 12 with an adhesive, hook and loop fastener, or other known methods suitable for the described application.

An elastic band 22 extends through the outermost ends of the side sections 20 and across the foot end 16 of the center section 12. The elastic band 22 serves to gather the surgical drape around the patient and the operating table during surgery. In some applications, it is desirable to utilize an X-ray fluoroscope machine. In this instance, the elastic band 22 and the side sections are sized to wrap around the X-ray machine, forming a pocket for the machine, thereby reducing the risk of contaminating the operating field. With the elastic margin wrapping around the machine, it is not necessary to have extra drape for this position since the drape forms a pocket around the machine.

The center section 12 is substantially T-shaped as shown including an upper body component 26 and a lower body component 28. The head-end of the upper body component 26 is formed by the horizontal part of the T. The horizontal portion of the “T” covers the patient's upper body, head and arms. The vertical leg of the T forming the lower body component 28 covers the torso and the lower extremities. In another embodiment, the head portion of the “T” may be opaque in its middle part but transparent at the sides to allow visualization of the arms through the drape during surgery. Such a design will permit monitoring of the arm position and also the various devices such as the intravenous fluid delivery system. In yet another embodiment, the entire drape may be made transparent.

The surgical drape may include a warming bladder positioned adjacent the center section 12. Specifically, the warming bladder may include an upper body bladder 30 adjacent the upper body component 26 of the center section 12 and a lower body bladder 32 positioned adjacent the lower body component 28 of the center section 12. The drape may or may not include the warming bladders 30, 32, and the bladders may be made separately and attached to the drape at the time of surgery. In an exemplary embodiment, the upper body bladder 30 is a T-shaped warming bladder preferably attached to an underside of the drape. The warming bladders 30, 32 may be manufactured as part of the drape or manufactured separately and then attached using any suitable attaching mechanism. The bladders may include a nozzle for attachment of a warm air circulator at any convenient location. The structure, use and operation of the nozzle and warming bladders are known, and further details will not be described

FIG. 2 is a close-up view of the operating window 18 and surgical fluid collection pockets 24. An adhesive tape 34 may be provided around the margins of the operating window 18 to seal off the operative field and secure a sterile field. The pockets 24 may be made as part of the drape, or may be removably attached adjacent the operating window 18 via a suitable fastener, such as a hook and loop fastener. The dimensions and the shape of the operating window 18 may vary, depending on the nature of the procedure. The aperture of the operating window 18 may be varied intra-operatively by various means, including cutting into the corners of the opening and folding or cutting the edges away; cutting out the backed-up adhesive periphery to enlarge the opening; or simply stretching the opening. The operating window 18 may have the entire area covered by an antibiotic impregnated adhesive sheet backed by a non-adhesive sheet, removable at the time of surgery. The pockets 24 may have a nozzle for the attachment of a suction tube to drain the pockets. The pockets 24 may be armed with a sealant entrance to prevent spillage of fluid or other material on to the floor at the end of the procedure.

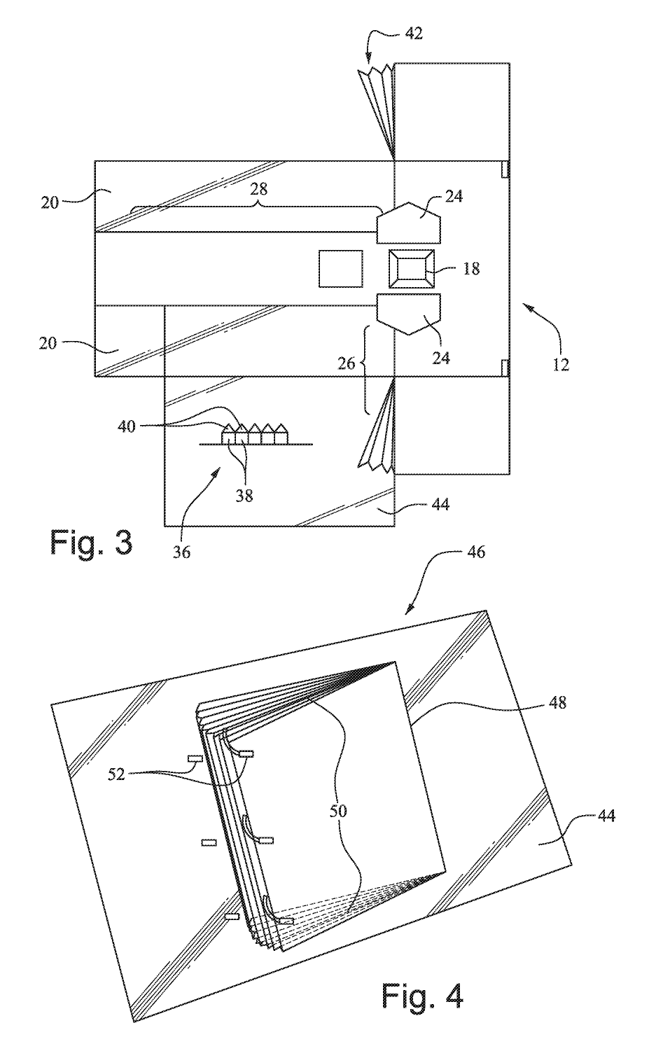

The surgical drape may additionally include an equipment manifold 36 attachable to the center section 12 or the side sections 20. The equipment manifold 36 is shown in plan view in FIG. 1, and a side view is shown in FIG. 3. The equipment manifold 36 includes a first plurality of compartments 38 for receiving surgical tubes and cables and a second plurality of compartments 40 for receiving surgical tools. As shown, the manifold 36 includes a first level with the first plurality of compartments 38 and a second level on top of the first level with the second plurality of compartments 40 (as shown in FIG. 3). One part of a removable fastener, such as a hook and fastener, may be secured to at least one of the center section 12 and the side sections 20, where the equipment manifold 36 includes another part of the removable fastener secured on an underside of the equipment manifold 36. The removable fastener provides for selective attachment of the equipment manifold 36 to the surgical drape.

FIG. 3 shows an alternative embodiment of the surgical drape, where similar reference numerals from the first embodiment shown in FIG. 1 are used for similar components. In the embodiment shown in FIG. 3, the upper body component 26 of the center section 12 may be provided with an arm extension drape 42 that is expandable to provide extra coverage to the arm, thus enhancing the sterile surgical field. The drape shown in FIG. 3 may also include a side extension drape 44 securable to one or both of the sides of the lower body component 28. The side extension drape 44 is preferably substantially transparent.

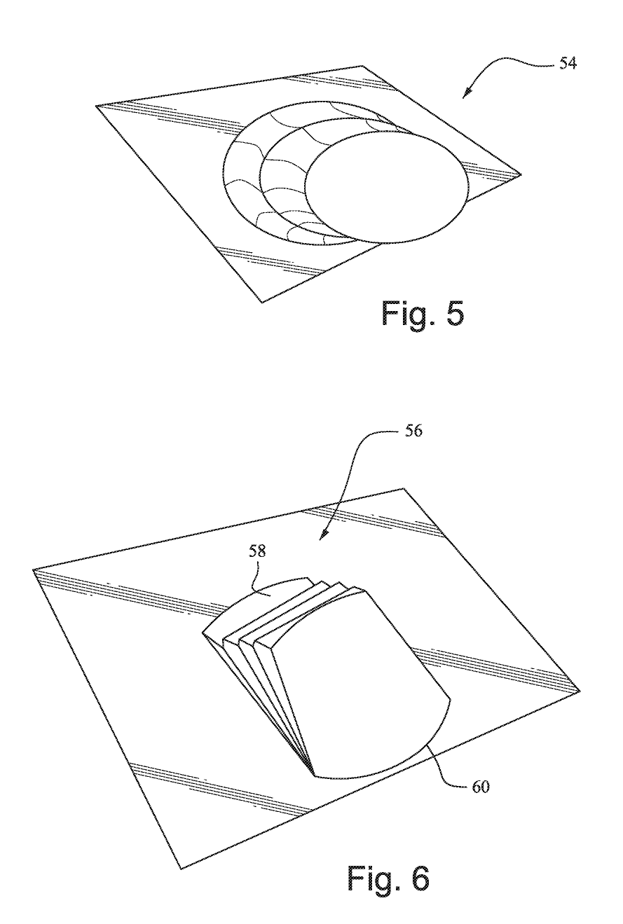

The side extension drape 44 provides a pocket into which the protruding arm of the X-ray fluoroscope can fit in a horizontal position. FIG. 4 shows a sideways expanding pouch 46 attached to the side extension drape 44. The pouch is connected to the side section 20 via a hinge 48 with folds 50 that concertina and are secured by a suitable fastener 52, such as a hook and loop fastener. FIG. 5 shows a variation on the expanding pouch as an out-pocketing 54 in the form of an expandable cylinder or the like with the center of its width corresponding to the center of the operating aperture for the X-ray machine. The out-pocketing 54 can be secured in a compressed orientation via a suitable fastener. FIG. 6 shows yet another alternative pouch 56 with an expandable proximal end 58 and a fixed distal end 60.

After sterile preparation of the surgical field, the drape is placed on the patient with the operative window over the proposed surgical site. The foot end 16 of the center section 12 and the side sections 20 wrap on either side of the patient and the operating table. When the X-ray fluoroscope machine is placed in a horizontal position, the elasticated side 20 of the drape forms a pocket around the machine keeping the field sterile. Alternatively, the side extension drape 44 is positioned over the machine. The upper section 26 of the drape covers the patient's head and shoulders. The pockets 24 on either side of the operating window 18 catch fluid from the surgical field. The transparency of the side sections 20 allows the surgeon to see foot pedals and the like on the floor during surgeries requiring the use of foot pedal activated tools (such as during minimally invasive spine surgery). The drape also advantageously enables the surgeon to see the X-ray fluoroscope as it is moved into the horizontal position, which serves to protect the patient from injury that may result from the machine pulling on devices attached to the patient.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A surgical drape comprising:
a center section including an operating window, the center section extending longitudinally from a head end to a foot end;
a side section positioned on each longitudinal side of the center section, the side sections being at least partially transparent;
an elastic band extending through outermost ends of the side sections and across the foot end of the center section;
at least one surgical fluid collection pocket disposed adjacent the operating window; and
a warming bladder positioned adjacent the center section.

2. A surgical drape according to claim 1, wherein the center section is T-shaped including an upper body component and a lower body component.

3. A surgical drape according to claim 2, wherein the warming bladder comprises an upper body bladder positioned adjacent the upper body component of the center section and a lower body bladder positioned adjacent the lower body component of the center section.

4. A surgical drape according to claim 2, wherein the upper body component comprises an arm extension drape that is expandable to ensure a sterile surgical field.

5. A surgical drape according to claim 4, further comprising a side extension drape securable to sides of the lower body component.

6. A surgical drape according to claim 1, wherein the center section comprises an arm extension drape that is expandable to ensure a sterile surgical field.

7. A surgical drape according to claim 1, further comprising a side extension drape securable to the longitudinal sides of the center section.

8. A surgical drape according to claim 7, wherein the side extension drape is substantially transparent.

9. A surgical drape according to claim 1, further comprising an expandable pocket part of or securable to at least one of the side sections, the expandable pocket being sized to cover a fluoroscopy machine adjacent the surgical drape.

10. A surgical drape according to claim 1, further comprising an equipment manifold attachable to the center section or the side sections, the equipment manifold including a first plurality of compartments for receiving surgical tubes and cables and a second plurality of compartments for receiving surgical tools.

11. A surgical drape according to claim 10, wherein the manifold comprises a first level with the first plurality of compartments and a second level on top of the first level with the second plurality of compartments.

12. A surgical drape according to claim 11, further comprising one part of a removable fastener secured to at least one of the center section and the side sections, wherein the equipment manifold comprises another part of the removable fastener secured on an underside of the equipment manifold and selectively attachable to the one part of the removable fastener.

13. A surgical drape according to claim 1, wherein the side sections are connected to the center section by sewing or gluing.

14. A surgical drape comprising:
a center section including an operating window, the center section extending longitudinally from a head end to a foot end;
a side section positioned on each longitudinal side of the center section, the side sections being at least partially transparent;
an elastic band extending through outermost ends of the side sections and across the foot end of the center section;
at least one surgical fluid collection pocket disposed adjacent the operating window;
an arm extension drape connected to the center section that is expandable to ensure a sterile surgical field; and
a side section with an elasticated margin that expands to wrap around a surgical table and a fluoroscopy machine in a horizontal position.

15. A surgical drape according to claim 14, wherein the center section is T-shaped including an upper body component and a lower body component.

16. A surgical drape according to claim 14, further comprising an equipment manifold attachable to the center section or the side sections, the equipment manifold including a first plurality of compartments for receiving surgical tubes and cables and a second plurality of compartments for receiving surgical tools.

17. A surgical drape according to claim 16, wherein the manifold comprises a first level with the first plurality of compartments and a second level on top of the first level with the second plurality of compartments.

18. A surgical drape according to claim 17, further comprising one part of a removable fastener secured to at least of the center section and the side sections, wherein the equipment manifold comprises another part of the removable fastener secured on an underside of the equipment manifold and selectively attachable to the one part of the removable fastener.

19. A surgical drape according to claim 14, further comprising a warming bladder positioned adjacent the center section.
